(19) United States
(12) Patent Application Publication
(10) Pub. No.: US 2024/0346393 A1
(43) Pub. Date: Oct. 17, 2024

(54) MATCHING DEVICE, MATCHING METHOD, AND RECORDING MEDIUM

(71) Applicant: NEC Corporation, Minato-ku, Tokyo (JP)

(72) Inventors: Yuan LUO, Tokyo (JP); Kosuke Nishihara, Tokyo (JP)

(73) Assignee: NEC Corporation, Minato-ku, Tokyo (JP)

(21) Appl. No.: 18/292,354

(22) PCT Filed: Aug. 4, 2021
(86) PCT No.: PCT/JP2021/028893

§ 371 Date: Jan. 26, 2024

(51) Int. Cl. G06Q 10/04; G06Q 50/10; G16H 40/20

(52) U.S. Cl. CPC G06Q 10/04; G06Q 50/10; G16H 40/20

(57) ABSTRACT

In a matching device, a priority setting means sets first priorities by arranging scores in a descending order which are assigned based on desired conditions of users who receive a service, and also sets second priorities by arranging scores in the descending order which are assigned based on desired conditions of providers. A priority interchange means sets at least third priorities in which the first priorities are interchanged or fourth priorities in which the second priorities are interchanged. A matching process means performs first and second matching processes based on combinations of the first to fourth priorities. A matching result selection means selects, as a final matching result, one of a first matching result acquired by the first matching process and a second matching result acquired by the second matching process.

### TECHNICAL FIELD

The present disclosure relates to a technique for performing matching capable of effectively utilizing resources in various services such as a medical service and the like.

### BACKGROUND ART

In various services such as a medical service and the like, a technique for performing matching which allows effective use of resources has been known.

Specifically, Non-Patent Document 1 discloses an example in which a deferred acceptance method is used as a matching method in a case of matching between children and childcare institutions.

Moreover, Non-Patent Document 2 discloses a technique for determining each priority for a plurality of request items each including a plurality of determination elements by setting a numerical group in which the plurality of determination elements and indexes concerning the plurality of determination elements are weighted in accordance with respective degrees of priority and by performing a predetermined calculation using the set numerical group.

### PRECEDING TECHNICAL REFERENCES

### Patent Document

Non-Patent Document 1: Yosuke Yasuda, “What can be done in the matching theory <First Part>”, [online], [Searched on May 14, 2021], internet<URL:https://note.com/yagena/n/nf9c9b87ce4a2>
Non-Patent Document 2: Tomoji Nagasawa, “Don't get lost anymore!“How to weight priorities“to determine the priority”, [online], [Searched on May 14, 2021], Internet<URL:https://backlog.com/ja/blog/priority-weighting-for-determining-the-order-of-priority/>

### SUMMARY

### Problem to be Solved by the Invention

One object of the present disclosure is to present an appropriate matching result in consideration of desires of both users of services and providers of the services.

### Means for Solving the Problem

According to an example aspect of the present disclosure, there is provided a matching device including:
a priority setting means configured to set first priorities by arranging scores in a descending order which are scores assigned based on desired conditions of a plurality of users who receive a service and which are assigned respectively to a plurality of providers serving the service, and also to set second priorities by arranging scores in the descending order which are scores assigned based on desired conditions of the plurality of providers and which are assigned respectively to the plurality of users;
a priority interchange means configured to set at least one of third priorities in which priorities of the plurality of providers are interchanged based on differences of the scores assigned in the first priorities and fourth priorities in which priorities of the plurality of users are interchanged based on differences of the scores assigned in the second priorities;
a matching process means configured to perform a first matching process based on combinations of the first priorities and the second priorities and also to perform a second matching process based on combinations of the third priorities and the second priorities, combinations of the first priorities and the fourth priorities, or combinations of the third priorities and the fourth priorities, as a matching process for matching the plurality of users and the plurality of providers; and
a matching result selection means configured to select, as a final matching result, one of a first matching result acquired by the first matching process and a second matching result acquired by the second matching process.

According to another example aspect of the present disclosure, there is provided a matching method including:
setting first priorities by arranging scores in a descending order which are scores assigned based on desired conditions of a plurality of users who receive a service and which are assigned respectively to a plurality of providers serving the service, and also setting second priorities by arranging scores in the descending order which are scores assigned based on desired conditions of the plurality of providers and which are assigned respectively to the plurality of users;
setting at least one of third priorities in which priorities of the plurality of providers are interchanged based on differences of the scores assigned in the first priorities and fourth priorities in which priorities of the plurality of users are interchanged based on differences of the scores assigned in the second priorities;
performing a first matching process based on combinations of the first priorities and the second priorities and also performing a second matching process based on combinations of the third priorities and the second priorities, combinations of the first priorities and the fourth priorities, or combinations of the third priorities and the fourth priorities, as a matching process for matching the plurality of users and the plurality of providers; and
selecting, as a final matching result, one of a first matching result acquired by the first matching process and a second matching result acquired by the second matching process.

According to a further example aspect of the present disclosure, there is provided a recording medium storing a program, the program causing a computer to perform a process including:
setting first priorities by arranging scores in a descending order which are scores assigned based on desired conditions of a plurality of users who receive a service and which are assigned respectively to a plurality of providers serving the service, and also setting second priorities by arranging scores in the descending order which are scores assigned based on desired conditions of the plurality of providers and which are assigned respectively to the plurality of users;
setting at least one of third priorities in which priorities of the plurality of providers are interchanged based on differences of the scores assigned in the first priorities and fourth priorities in which priorities of the plurality of users are interchanged based on differences of the scores assigned in the second priorities;
performing a first matching process based on combinations of the first priorities and the second priorities and also performing a second matching process based on combinations of the third priorities and the second priorities, combinations of the first priorities and the fourth priorities, or combinations of the third priorities and the fourth priorities, as a matching process for matching the plurality of users and the plurality of providers; and
selecting, as a final matching result, one of a first matching result acquired by the first matching process and a second matching result acquired by the second matching process.

### Effect of the Invention

According to the present disclosure, it is possible to present an appropriate matching result in consideration of desires of both users of services and providers of the services.

### BRIEF DESCRIPTION OF THE DRAWINGS

FIG. 1 illustrates an example of a configuration of a matching system including a server device according to a first example embodiment.

FIG. 2 is a block diagram illustrating a hardware configuration of the server device.

FIG. 3 is a block diagram illustrating a functional configuration of the server device according to the first example embodiment.

FIG. 4 is a flowchart for explaining operations and the like of the server device according to the first example embodiment.

FIG. 5 is a block diagram illustrating a functional configuration of the server device according to a second example embodiment.

FIG. 6 is a flowchart for explaining of operations and the like of the server device according to the second example embodiment.

FIG. 7 is a block diagram illustrating a functional configuration of a server device according to a third example embodiment.

FIG. 8 is a flowchart for explaining of operations and the like of the server device according to the third example embodiment.

FIG. 9 is a block diagram illustrating a functional configuration of a server device according to a fourth example embodiment.

FIG. 10 is a flowchart for explaining of operations and the like of the server device according to the fourth example embodiment.

### EXAMPLE EMBODIMENTS

In the following, preferred example embodiments in the present disclosure will be described with reference to the accompanying drawings.

### First Example Embodiment

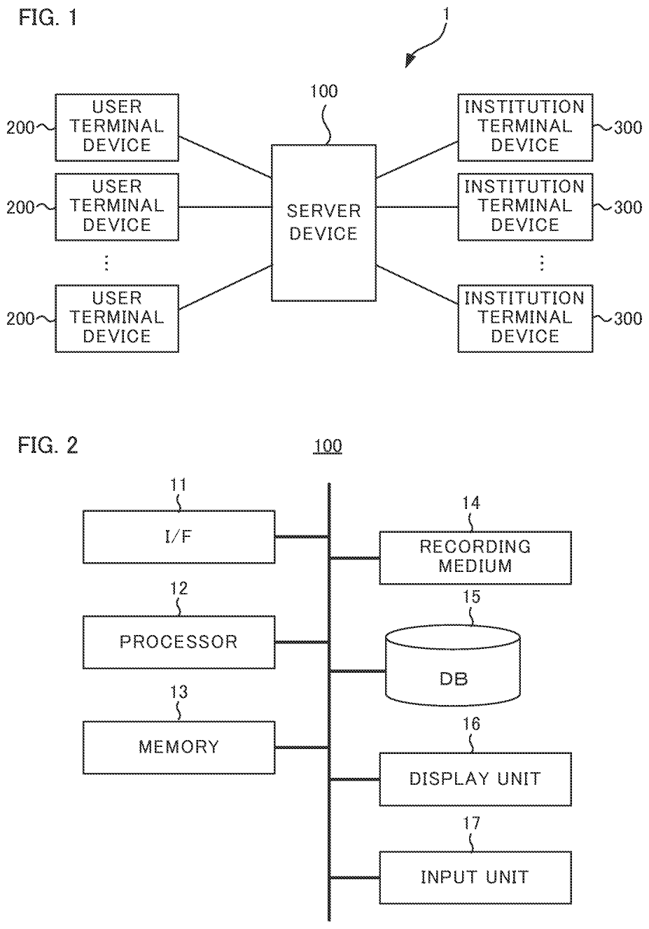

FIG. 1 is a diagram illustrating an example of a configuration of a matching system including a server device according to a first example embodiment.

A matching system 1 includes a server device 100, a plurality of user terminal devices 200, and a plurality of institution terminal devices 300 as depicted in FIG. 1.

The server device 100 includes a function as a matching device in the matching system 1. Also, the server device 100 is formed to be able to communicate with each of the plurality of user terminal devices 200 and each of the plurality of institution terminal devices 300. Moreover, the server device 100 is formed to perform a matching process (details are described below) for matching a plurality of users and a plurality of institutions based on information sent from each of the plurality of user terminal devices 200 and information sent from each of the plurality of institution terminal devices 300. Furthermore, the server device 100 is formed to send a final matching result obtained through a process such as a matching process or the like to the user terminal device 200 or the institution terminal device 300 serving as senders of the information.

The user terminal device 200 is associated with a user who desires to use a service provided by each institution of which the institution terminal device 300 is provided. In other words, a plurality of user terminal devices 200 are respectively associated with a plurality of users receiving services. Moreover, the user terminal device 200 includes a function for communicating with the server device 100, a function for inputting information to be transmitted to the server device 100, and a function for displaying information received from the server device 100. Specifically, the user terminal device 200 is formed by a device such as a personal computer, a smartphone, a tablet type computer, and the like, for instance.

The institution terminal device 300 is provided in an institution for providing a service to each user among the users associated with the user terminal device 200. In other words, the plurality of institution terminal devices 300 are respectively associated with the plurality of providers for providing the service. Moreover, each institution terminal device includes a function for communicating with the server device 100, a function for inputting information to be transmitted to the server device 100, and a function for displaying information received from the server device 100. Specifically, the institution terminal device 300 is formed by a device such as a personal computer, a smartphone, and a tablet type computer, for instance.

FIG. 2 is a block diagram illustrating a hardware configuration of the server device 100.

As illustrated in FIG. 2, the server device 100 includes an interface (I/F) 11, a processor 12, a memory 13, a recording medium 14, a database (DB) 15, a display unit 16, and an input unit 17.

The interface 11 is formed to be able to communicate with the plurality of user terminal devices 200 and the plurality of institution terminal devices 300. That is, information transmitted from each user terminal device 200 and each institution terminal device 300 is received at the interface 11. Also, each process result of the matching process is sent to a corresponding user terminal device 200 and a corresponding institution terminal device 300 through the interface 11.

The processor 12 is formed by a CPU (Central Processing Unit) or the like, and controls the entire server device 100 by executing programs prepared in advance. Note that the processor 12 may be a GPU (Graphics Processing Unit) or a FPGA (Field-Programmable Gate Array). The processor 12 also performs a matching process and processes concerning the matching process using information received at the interface 11.

The memory 13 is formed by a ROM (Read Only Memory) and a RAM (Random Access Memory). The memory 13 is also used as a working memory during executions of various processes by the processor 12.

The recording medium 14 is a non-volatile and non-transitory recording medium such as a disk-like recording medium or a semiconductor memory and is formed to be detachable from the server device 100. Moreover, the recording medium 14 stores various programs executed by the processor 12. When the server device 100 executes various processes, the program stored in the recording medium 14 is loaded into the memory 13 and executed by the processor 12. Furthermore, the recording medium 14 stores information used in the execution of the matching process.

The DB 15 stores the process results and the like of the matching process as needed.

The display unit 16 is constituted by a display device such as a liquid crystal monitor, for instance. In addition, the display unit 16 displays information such as the process result of the matching process if necessary.

The input unit 17 includes an input device such as a keyboard, a mouse, or a touch panel.

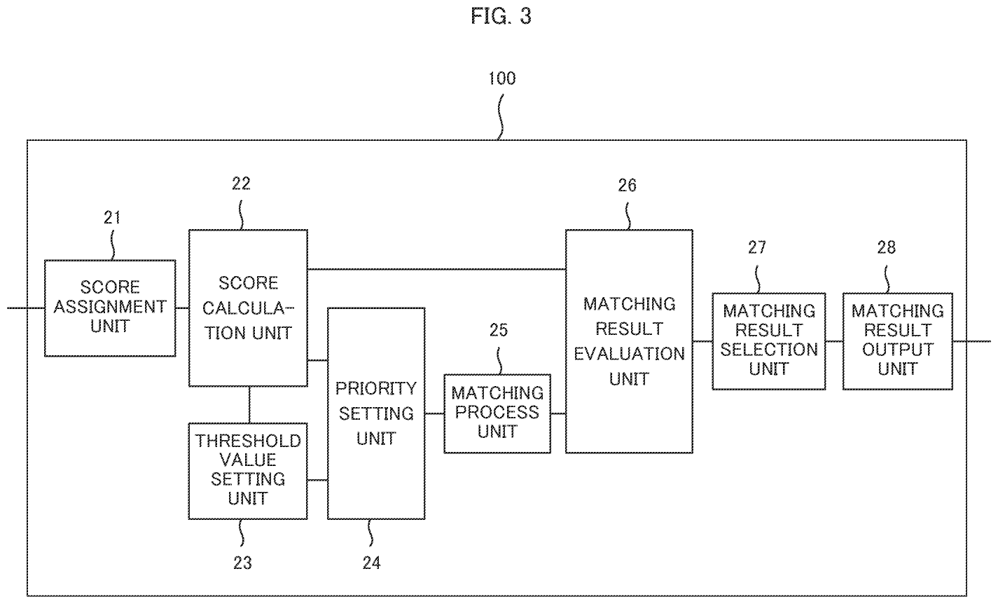

FIG. 3 is a block diagram illustrating a functional configuration of the server device 100 according to the first example embodiment.

As depicted in FIG. 3, the server device 100 includes a score assignment unit 21, a score calculation unit 22, a threshold value setting unit 23, a priority setting unit 24, a matching process unit 25, a matching result evaluation unit 26, a matching result selection unit 27, and a matching result output unit 28.

The score assignment unit 21 extracts a plurality of user desired items (desired conditions) included in the information transmitted from each user terminal device 200, and assigns an institution evaluation score Fe for each of the plurality of user desired items by applying a user evaluation reference (described later for a specific example) to each of a plurality of extracted user desired items.

The score assignment unit 21 extracts a plurality of institution desired items (desired conditions) included in the information transmitted from the institution terminal device 300, and assigns a user evaluation score Ue for each of the plurality of institution desired items by applying the institution evaluation reference to each of a plurality of extracted institution desired items (described later for a specific example).

The score calculation unit 22 calculates each user priority score Fp which is a value that is increased or decreased in accordance with an element such as whether or not the user desires to receive a service in the institution to be evaluated based on the user evaluation reference described above, by performing a predetermined calculation (described later for a specific example) using each institution evaluation score Fe assigned by the score assignment unit 21. In other words, the score calculation unit 22 calculates respective user priority scores Fp corresponding to the plurality of institutions to be evaluated by one user associated with the user terminal device 200.

The score calculation unit 22 calculates each institution priority score Up which is a value to be increased or decreased in accordance with an element such as whether or not a necessary and sufficient service can be provided with respect to the user to be evaluated based on the institution evaluation reference, by performing a predetermined calculation (described later for a specific example) using the user evaluation score Ue assigned by the score assignment unit 21. In other words, the score calculation unit 22 calculates respective institution priority scores Up corresponding to the plurality of users to be evaluated by one institution in which the institution terminal device 300 is provided.

The threshold value setting unit 23 sets, as a user threshold value Ft, a value of a score obtained by multiplying the highest value in the user priority scores Fp calculated in the score calculation unit 22 by an allowable ratio Fa corresponding to a ratio allowed as an approximate range among the plurality of user priority scores Fp obtained as calculation results of the score calculation unit 22.

Specifically, for instance, the threshold value setting unit 23 sets the user threshold value Ft as 5 points in a case where the highest value in the user priority scores Fp is 100 points and the allowable ratio Fa is 5%.

The threshold value setting unit 23 sets, as an institution threshold value Ut, a value of the score obtained by multiplying the highest value in the institution priority scores Up calculated in the score calculation unit 22 by an allowable ratio Ua corresponding to a ratio allowed as an approximate range in the plurality of institution priority scores Up obtained as calculation results of the score calculation unit 22.

Specifically, for instance, the threshold value setting unit 23 sets the institution threshold value Ut as 5 points in a case where the highest value in the institution priority scores Up is 100 points and the allowable ratio Ua is 5%.

Note that by setting the allowable ratio Fa to 0, it is assumed that the threshold value setting unit 23 is capable of setting the user threshold value Ft as 0 points. Moreover, by setting the allowable ratio Ua to 0, it is assumed that the threshold value setting unit 23 is capable of setting the institution threshold value Ut as 0 points.

The priority setting unit 24 sets the user priority including the priority in accordance with an order of the institutions desired to be used by the user based on the plurality of user priority scores Fp obtained as the calculation result of the score calculation unit 22 and the user threshold value Ft set by the threshold value setting unit 23.

In detail, for instance, in a case where the user threshold value Ft is set to X points by the threshold value setting unit 23, the priority setting unit 24 presumes that among the plurality of user priority scores Fp obtained as the calculation results of the score calculation unit 22, the priorities of the institutions corresponding to respective user priority scores Fp are mutually interchangeable in which each difference of scores is in a range of the X points or less. Next, the priority setting unit 24 sets, as user priorities, priorities ordered by the user priority scores Fp in a higher order and priorities depending on combinations of the user priority scores Fp which are mutually interchangeable as estimated as described above, based on the plurality of user priority scores Fp acquired as the calculation result of the score calculation unit 22. Note that in a case of X=0, the priority setting unit 24 sets, as the user priorities, priorities ordered in which the user priority scores Fp are arranged in the higher order.

The priority setting unit 24 sets the institution priorities including priorities in accordance with the order of the users who desire to accept the institutions based on the plurality of institution priority scores Up obtained as the calculation results of the score calculation unit 22 and the institution threshold value Ut set by the threshold value setting unit 23.

Specifically, for instance, in a case where the institution threshold value Ut is set to Y points by the threshold value setting unit 23, the priority setting unit 24 presumes that among the plurality of institution priority scores Up obtained as the calculation results of the score calculation unit 22, the priorities of the users corresponding to respective institution priority scores Up are mutually interchangeable in which each difference of scores is in a range of the Y points or less. Next, the priority setting unit 24 sets, as institution priorities, priorities of arranging the institution priority scores Up in a higher order and priorities corresponding to combinations of the institution priority scores Up which are mutually interchangeable as estimated as described above, based on the plurality of institution priority scores Up acquired as the calculation result of the score calculation unit 22. Incidentally, the priority setting unit 24 sets, as the institution priorities, priorities ordered in which the user priority scores Fp are arranged in the higher order in a case of Y=0.

That is, the priority setting unit 24 includes a function as a priority setting means, sets the user priorities (first priorities) by arranging the user priority scores Fp in a descending order which are scores assigned in accordance with respective desired conditions of the plurality of users who receive the services and are respectively assigned to the plurality of providers who provide the services, and also sets the institution priorities (second priorities) by arranging the institution priority scores Up in the descending order which are assigned in accordance with respective desired conditions of the plurality of providers and are respectively assigned to the plurality of users. Moreover, the priority setting unit 24 includes a function as a priority interchange means, and sets at least one of priorities (third priorities) in which the priorities of the providers are interchanged based on differences among respective scores assigned in the user priorities or priorities (fourth priorities) in which priorities of the users are interchanged based on differences among respective scores assigned in the institution priorities. Furthermore, the third priorities are set by interchanging the priorities of the providers whose differences in the scores assigned in the user priorities fall within a range of the user threshold values Ft. In addition, the fourth priorities are set by interchanging the priorities of the users for which differences in scores assigned in the institution priorities fall within the institution threshold value Ut.

The matching process unit 25 performs the matching process based on respective priorities included in the user priorities and the institution priorities which are set by the priority setting unit 24.

Note that, in the description of the present disclosure, unless otherwise noted, the matching process unit 25 performs the matching process using a deferred acceptance method for combinations of all priorities included in the user priorities and the institution priorities which are set by the priority setting unit 24.

The matching result evaluation unit 26 calculates matching result evaluation values each which indicates a value representing high or low in the evaluation of the matching result by performing a process based on the plurality of user priority scores Fp calculated by the score calculation unit 22, the plurality of institution priority scores Up calculated by the score calculation unit 22, and the matching result obtained by the matching process unit 25.

The matching result selection unit 27 selects one matching result evaluation value from among the matching result evaluation values calculated by the matching result evaluation unit 26, and acquires one matching result associated with the selected matching result evaluation value as the final matching result.

The matching result output unit 28 outputs (sends) the final matching result obtained by the matching result selection unit 27 to the corresponding user terminal device 200 and the corresponding institution terminal device 300.

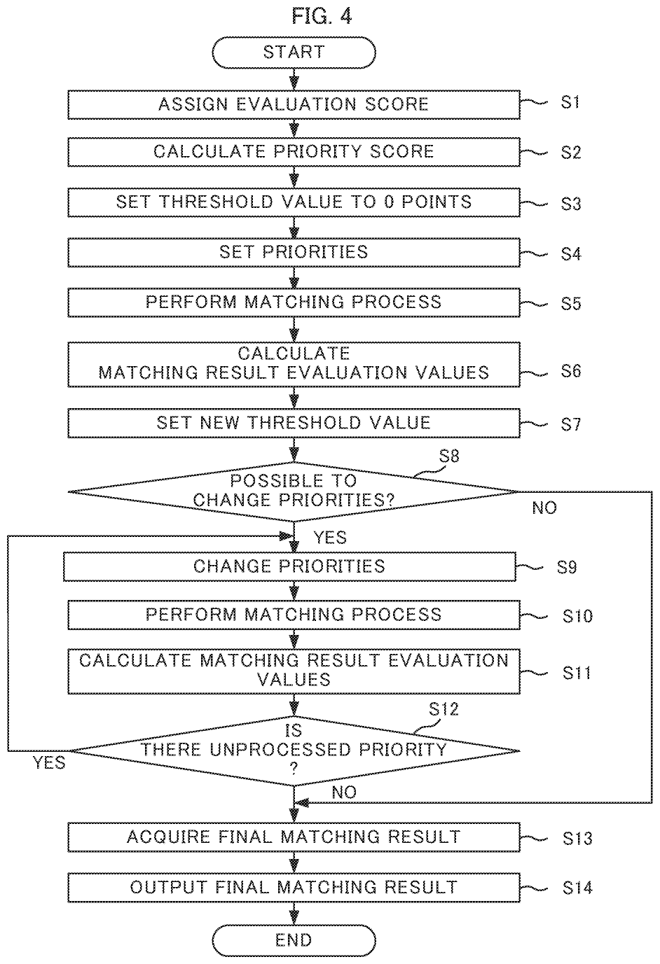

Subsequently, operations in the present example embodiment will be described with reference to a flowchart in FIG. 4. FIG. 4 is the flowchart for explaining operations and the like of the server device according to the first example embodiment.

Note that hereinafter, descriptions will be based on an example of a case where the institution terminal devices 300 are installed in medical institutions as the institutions providing medical services, and the user terminal devices 200 are associated to information of patients who are the users receiving the medical services. Moreover, hereinafter, the institution terminal devices 300 are provided respectively to three institutions: an institution P1, an institution P2, and an institution P3, the user terminal devices 200 are provided respectively for five patients: a patient Q1, a patient Q2, a patient Q3, a patient Q4, and a patient Q5. Furthermore, in the following, various scores are expressed as one of the scores from 0 to 100.

The patient operates the user terminal device 200 to input information including the plurality of user desired items regarding the evaluation of the medical institutions from which the medical services are provided. The user terminal device 200 transmits information input by the patient to the server device 100.

For instance, the plurality of user desired items described above may include items such as a distance between an address of the patient and the medical institution, a preferred fee for the medical service, a hospitalization coordination time, whether daily necessities are allowed or not in the hospital, reputation of the medical institution, and the like. Moreover, the plurality of user desired items described above may include, for instance, an item regarding a user preference with respect to the medical institution in a case of receiving the medical service. That is, the plurality of user desired items may include one or more items regarding the user preference in the case of receiving the medical service.

A medical personnel such as a medical doctor or the like operates the institution terminal device 300 to input information including the plurality of institution desired items regarding the evaluation of the patient whom the medical service is provided. The institution terminal device 300 transmits the information input by the medical professional to the server device 100.

The plurality of institution desired items may include, for instance, items such as the number of available beds, a type of a medical condition, a severity of illness, and the like. Moreover, for instance, the plurality of institution desired items described above may also include items regarding preference of the medical institution for accepting the patient. Furthermore, the plurality of institution desired items described above may include, for instance, items concerning the desire of the medical institution for the users. In other words, it is sufficient that the plurality of institution desired items described above includes one or more items regarding an ability to accept the patients in a case of accepting the patient by the medical institution.

The score assignment unit 21 extracts the plurality of user desired items included in the information transmitted from each user terminal device 200, and assigns the institution evaluation scores Fe corresponding to the plurality of user desired items by applying the user evaluation reference to each of the plurality of user desired items which are extracted (step S1 in FIG. 4).

In the user evaluation reference, values for a plurality of scores corresponding to respective input values for the plurality of user desired items are set in advance. Therefore, for instance, with respect to an input value DV that is less than 5 km for the distance between the address of the patient extracted as the user desired item and the medical institution, the score assignment unit 21 assigns 10 points as the institution evaluation score Fe. Moreover, for instance, with respect to the above-described input value DV is equal to or more than 5 km and less than 10 km, the score assignment unit 21 assigns 5 points as the institution evaluation score Fe. Furthermore, for instance, with respect to the above-described input value DV is equal to or more than 10 km, the score assignment unit 21 assigns 0 points as the institution evaluation score Fe.

The score assignment unit 21 extracts the plurality of institution desired items included in the information transmitted from the institution terminal device 300, and assigns the user evaluation scores Ue corresponding to the plurality of institution desired items by applying the institution evaluation reference to each of the plurality of institution desired items which are extracted (step S1 in FIG. 4).

In the institution evaluation reference, values for a plurality of scores corresponding to respective input values for the plurality of institution desired items are set in advance. Therefore, for instance, in a case where an input value MV of the severity of illness extracted as the institution desired item indicates a mild illness, the score assignment unit 21 assigns 10 points as the user evaluation score Ue. Moreover, for instance, with respect to the input value MV described above that indicates a moderate illness, the score assignment unit 21 assigns 5 points as the user evaluation score Ue. Furthermore, for instance, with respect to the input value MV described above that indicates a serious illness, the score assignment unit 21 assigns 0 points as the user evaluation score Ue. Note that such a method for assigning the user evaluation score Ue is an example of preferentially accepting patients with less severe illnesses, the method may be replaced by another method for assigning the score. Specifically, for instance, a high user evaluation score Ue may be assigned to a patient with a higher severity, and a low user evaluation score Ue may be assigned to a patient with a less severity.

The score calculation unit 22 calculates the user priority score Fp by performing a predetermined calculation based on the institution evaluation scores Fe assigned by a process of step S1 in FIG. 4 and weight coefficients WF preset as values corresponding to the plurality of user desired items extracted by the score assignment unit 21 (step S2 in FIG. 4).

Specifically, the score calculation unit 22 calculates the user priority score Fp by applying the weight coefficient WFi and the institution evaluation score Fei corresponding to an i-th item among n user desired items to a right side of the following formula (1).

$$\begin{matrix}[ Math 1 ] & \\ Fp = {\sum }_{i = 1}^{n} WFi \times Fei & ( 1 )\end{matrix}$$

That is, according to a process of step S2 in FIG. 4 as described above, for instance, the score calculation unit 22 calculates the user priority score Fp as 71 points when i=10, WF1 to WF9=1.0, WF10=0.5, Fe1=10, Fe2=5, Fe3=10, Fe4=0, Fe5=8, Fe6=10, Fe7=9, Fe8=7, Fe9=8, and Fe10=8.

Moreover, according to the process of step S2 in FIG. 4 as described above, the score calculation unit 22 calculates the user priority score Fp1 of the institution P1, the user priority score Fp2 of the institution P2, and the user priority score Fp3 of the institution P3 as respective user priority scores for the patients Q1 to Q5.

The score calculation unit 22 calculates respective institution priority scores Up by performing a predetermined calculation based on the user evaluation scores Ue assigned by the process of step S1 in FIG. 4 and weight coefficients WU defined in advance as values corresponding to the plurality of institution desired items extracted by the score assignment unit 21 (step S2 in FIG. 4).

Specifically, the score calculation unit 22 calculates one institution priority score Up by applying a weight coefficient WUk and a user evaluation score Uek corresponding to the k-th item among n institution desired items to a right side of the following formula (2).

$$\begin{matrix}[ Math 2 ] & \\ Up = {\sum }_{i = 1}^{n} WUk \times Uek & ( 2 )\end{matrix}$$

That is, according to the process of step S2 in FIG. 4 as described above, for instance, the score calculation unit 22 calculates the institution priority score Up as 71 points when k=10, WU1 to WU9=1.0, WU10=0.5, Ue1=10, Ue2=5, Ue3=10, Ue4=0, Ue5=8, Ue6=10, Ue7=9, Ue8=7, Ue9=8, and Ue10=8.

In addition, according to the process of step S2 depicted in FIG. 4 as described above, as respective institution priority scores for the institutions P1 to P3, the score calculation unit 22 calculates the institution priority score Up1 of the patient Q1, the institution priority score Up2 of the patient Q2, the institution priority score Up3 of the patient Q3, the institution priority score Up4 of the patient Q4, and the institution priority score Up5 of the patient Q5.

The threshold value setting unit 23 sets the user threshold value Ft and institution threshold value Ut to 0 points by setting the allowable ratio Fa=Ua=0 (step S3 in FIG. 4).

The priority setting unit 24 sets, as the user priorities, priorities ordered in the descending order of the user priority scores Fp calculated by the process of step S2 in FIG. 4 in response to setting the user threshold value Ft (=0 points) by a process of step S3 in FIG. 4 (step S4 in FIG. 4). Moreover, the priority setting unit 24 sets, as the institution priorities, priorities ordered in the descending order of the institution priority scores Up calculated by the process of step S2 in FIG. 4 in response to setting the institution threshold value Ut (=0 points) by the process of step S3 in FIG. 4 (step S4 in FIG. 4).

The matching process unit 25 performs the matching process described below based on the user priorities and the institution priorities set by a process of step S4 in FIG. 4 (step S5 in FIG. 4). The matching process unit 25 stores the matching result obtained by a process of step S5 in FIG. 4 in the DB 15.

The matching result evaluation unit 26 calculates the matching result evaluation values by performing the process described below based on the plurality of user priority scores Fp and the plurality of institution priority scores Up calculated by the process of step S2 in FIG. 4 and the matching result obtained by the process of step S5 in FIG. 4 (step S6 in FIG. 4). Also, the matching result evaluation unit 26 associates the matching result evaluation values calculated by a process of step S6 in FIG. 4 with the matching result stored in the DB 15 by the process of step S5 in FIG. 4.

The threshold value setting unit 23 sets a new threshold value by changing at least one of the user threshold value Ft and the institution threshold value Ut (step S7 in FIG. 4).

Here, in the following description of the present example embodiment, unless otherwise noted, a case will be described in which the user threshold value Ft is set to 5 points and the institution threshold value Ut is set to 0 points by the process step S7 in FIG. 4.

The priority setting unit 24 determines whether it is possible or not to change the user priorities set in step S4 in FIG. 4 to other priorities based on the plurality of user priority scores Fp calculated by the process of step S2 in FIG. 4 and the user threshold value Ft set by a process of step S7 in FIG. 4 (step S8 in FIG. 4).

Specifically, in step S8 in FIG. 4, among the plurality of user priority scores Fp calculated by the process of step S2 in FIG. 4, the priority setting unit 24 detects whether or not there are the user priority scores in which each difference in scores falls in a range of 5 points or less.

When it is not possible to change the user priorities set in step S4 in FIG. 4 to other priorities (S8: NO), the priority setting unit 24 causes the matching result selection unit 27 to perform a process of step S13 in FIG. 4 described later. Moreover, when it is possible to change the user priorities set in step S4 in FIG. 4 to other priorities (S8: YES), the priority setting unit 24 performs a process of step S9 in FIG. 4 described later.

Specifically, the priority setting unit 24 determines that it is not possible to change the user priorities set in step S4 in FIG. 4 to other priorities in a case where there are no user priority scores Fp for which each difference of scores falls in the range of 5 points or less among the plurality of user priority scores Fp calculated by the process of step S2 in FIG. 4, and causes the matching result selection unit 27 to perform the process of step S13 in FIG. 4 described below. The priority setting unit 24 determines that it is possible to change the user priorities set in step S4 in FIG. 4 to other priorities in a case of existing one or more differences of scores which fall in the range of 5 points or less among the plurality of user priority scores Fp calculated by the process of step S2 in FIG. 4, and performs the process of step S9 in FIG. 4 described below.

The priority setting unit 24 changes the user priorities which are currently set, to unprocessed priorities in which the calculation of the matching result evaluation values by a process of step S11 in FIG. 4 described later has not been performed, by mutually interchanging priorities of the institutions corresponding to respective user priority scores Fp of which each difference of scores falls within a range of the user threshold value Ft set by the process of step S7 in FIG. 4 among the priorities of user priority scores Fp calculated by the process of step S2 in FIG. 4 (step S9 in FIG. 4).

In the following description of the present example embodiment, a case will be described with reference to a specific example in which by the process of step S2 in FIG. 4, the user priority scores for the patient Q1 are calculated and resulted in Fp1=88 points, Fp2=90 points, and Fp3=70 points, the user priority scores for the patients Q2, Q3 and Q5 are calculated and resulted in Fp1=90 points, Fp2=80 points, and Fp3=70 points, and the user priority scores for the patient Q4 are calculated and resulted in Fp1=80 points, Fp2=90 points, and Fp3=80 points. Moreover, in subsequent descriptions of the present example embodiment, a case will be described with reference to a specific example in which by the process of step S2 in FIG. 4, the institution priority scores for the institution P1 are calculated and resulted in Up1=90 points, Up2=70 points, Up3=80 points, Up4=60 points, and Up5=50 points, the institution priority scores for the institution P2 are calculated and resulted in Up1=80 points, Up2=70 points, Up3=90 points, Up4=50 points, and Up5=60 points, and the institution priority scores for the institution P3 are calculated and resulted in Up1=70 points, Up2=90 points, Up3=60 points, Up4=50 points, and Up5=80 points.

In the specific example described above, the priority setting unit 24 sets priorities expressing an order of the institution P2->the institution P1->the institution P3 as the user priorities for the patient Q1 by the process of step S4 in FIG. 4. In addition, in a case of the specific example described above, the priority setting unit 24 sets priorities expressing an order of the institution P1->the institution P2->the institution P3 as the user priorities for the patients Q2, Q3 and Q5 by the process of step S4 in FIG. 4. Furthermore, in the case of the specific example described above, the priority setting unit 24 sets priorities expressing an order of the institution P2->the institution P1->the institution P3 as the user priorities for the patient Q4 by the process of step S4 in FIG. 4.

In the specific example described above, the priority setting unit 24 sets priorities expressing an order of the patient Q1->the patient Q3->the patient Q2->the patient Q4->the patient Q5 as the institution priorities for the institution P1 by the process of step S4 in FIG. 4. In addition, in the case of the specific example described above, the priority setting unit 24 sets priorities expressing an order of the patient Q3->the patient Q1->the patient Q2->the patient Q5->the patient Q4 as the institution priorities for the institution P2 by the process of step S4 in FIG. 4. In the case of the specific example described above, the priority setting unit 24 sets priorities expressing an order of the patient Q2->the patient Q5->the patient Q1->the patient Q3->the patient Q4 as the institution priorities for the institution P3 by the process of step S4 in FIG. 4.

In the case of the specific example described above, the priority setting unit 24 determines in step S8 in FIG. 4 that it is possible to change the user priorities because the user priority scores Fp1 and Fp2 for the patient Q1 fall in 5 points or less.

In the case of the specific example described above, by mutually interchanging the priorities for the institution P1 and the institution P2 in step S9 in FIG. 4, the priority setting unit 24 changes the user priorities set in step S4 in FIG. 4 to the unprocessed priorities in which the matching result evaluation values have not been calculated by the process of step S11 in FIG. 4.

More specifically, the priority setting unit 24 changes the user priorities for the patient Q1 from the order of the institution P2->the institution P1->the institution P3 to the order of the institution P1->the institution P2->the institution P3, by the process of step S9 in FIG. 4.

The matching process unit 25 includes a function as the matching process means, and performs the matching process based on the user priorities changed by the process of step S9 in FIG. 4 and the institution priorities set by the process of step S4 in FIG. 4 (step S10 in FIG. 4). Moreover, the matching process unit 25 stores the matching result obtained by the process of step S10 in FIG. 4 in the DB 15.

Here, according to the present example embodiment, in step S5 and step S10 in FIG. 4, it is sufficient that at least one of a process using a first matching method which prioritizes the desires of the medical institutions or a process using a second matching method which prioritizes desires of the patients is performed.

Details of the first matching method described above will be described below.

The matching process unit 25 performs the matching process by the deferred acceptance method based on the user priorities (the first priorities) and the institution priorities (second priorities) set by the process of step S4 in FIG. 4, and thus acquires a matching result M11 in which the patients Q3 and Q2 are matched to the institution P1, the patients Q1 and Q5 are matched to the institution P2, and the patient Q4 is matched to the institution P3. That is, the matching process unit 25 acquires the matching result M11 by a matching process in step S5 (first matching process) in FIG. 4.

In addition, the matching process unit 25 performs the matching process by the deferred acceptance method based on user priorities (third priorities) changed by the process of step S9 in FIG. 4 and the institution priorities (the second priorities) set by the process of step S4 in FIG. 4, and thus acquires a matching result M12 in which the patients Q1 and Q2 are matched to the institution P1, the patients Q3 and Q5 are matched to the institution P2, and the patient Q4 is matched to the patient P3. That is, the matching process unit 25 acquires the matching result M12 by a matching process of step S10 (second matching process) in FIG. 4.

Details of the second matching method described above will be described below.

Based on the user priorities (first priorities) and the institution priorities (second priorities) set by the process of step S4 in FIG. 4, the matching process unit 25 performs the matching process by the deferred acceptance method, and thus acquires a matching result M21 in which the institution P1 is matched for the patients Q2 and Q3, the institution P2 is matched to the patients Q1 and Q5, and the institution P3 is matched to the patient Q4. That is, the matching process unit 25 acquires the matching result M21 by the matching process in step S5 (first matching process) in FIG. 4.

In addition, the matching process unit 25 performs the matching process by the deferred acceptance method based on the user priorities (the third priorities) changed by the process of step S9 in FIG. 4 and the institution priorities (the second priorities) set by the process of step S4 in FIG. 4, and thus acquires a matching result M22 in which the institution P1 is matched to the patients Q1 and Q2, the institution P2 is matched to the patients Q3 and Q5, and the institution P3 is matched to the patient Q4. That is, the matching process unit 25 acquires the matching result M22 by the matching process of step S10 (second matching process) in FIG. 4.

The matching result evaluation unit 26 calculates each matching result evaluation value by performing a process described later based on the plurality of user priority scores Fp and the plurality of institution priority scores Up which are calculated by the process of step S2 in FIG. 4 and the matching result obtained by the matching process unit 25 (step S11 in FIG. 4). The matching result evaluation unit 26 associates the matching result evaluation values calculated by the process of step S11 in FIG. 4 with the matching result stored in the DB 15 by the process of step S10 in FIG. 4.

Here, in the present example embodiment, as each of the processes for calculating the matching result evaluation values in step 6 and step 11 in FIG. 4, it is sufficient that at least one of a first evaluation value calculation process for calculating each value associated with the matching result of the first matching process described above, a second evaluation value calculation process for calculating each value associated with the matching result of the second matching process described above, and a third evaluation value calculation process for calculating each value associated with both the matching results of the first and second matching processes described above is performed.

Details of the first evaluation value calculation process described above will be described below.

Based on the matching result M11 obtained by the process of step S5 in FIG. 4, the matching result evaluation unit 26 extracts Up3 and Up2 for the institution P1, and Up1 and Up5 for the institution P2, and Up4 for the institution P3 from among the plurality of institution priority scores calculated by the process of step S2 in FIG. 4. Then, the matching result evaluation unit 26 calculates a value obtained by summing the institution priority scores extracted as described above as a matching result evaluation value A11 associated with the matching result M11. That is, the matching result evaluation unit 26 calculates the matching result evaluation value A11 by performing the process described above as the process of step S6 in FIG. 4, and associates the calculated matching result evaluation value A11 to the matching result M11.

Based on the matching result M12 obtained by the process of step S10 in FIG. 4, the matching result evaluation unit 26 extracts Up1 and Up2 for the institution P1, and Up3 and Up5 for the institution P2, and Up4 for the institution P3 from among the plurality of institution priority scores calculated by the process of step S2 in FIG. 4. After that, the matching result evaluation unit 26 calculates a value obtained by summing the institution priority scores extracted as described above, as a matching result evaluation value A12 associated with the matching result M12. That is, the matching result evaluation unit 26 calculates the matching result evaluation value A12 by performing the process described above as the process of step S11 in FIG. 4, and associates the calculated matching result evaluation value A12 with the matching result M12.

Details of the second evaluation value calculation process described above will be described below.

The matching result evaluation unit 26 extracts Fp1 for the patient Q2 and Q3, Fp2 for the patient Q1 and Q5, and Fp3 for the patient Q4 from among the plurality of user priority scores calculated by the process of step S2 in FIG. 4 based on the matching result M21 obtained by the process of step S10 in FIG. 4. Next, the matching result evaluation unit 26 calculates a value obtained by summing the user priority scores extracted as described above as a matching result evaluation value A21 associated with the matching result M21. That is, the matching result evaluation unit 26 calculates the matching result evaluation value A21 by performing the process described above as the process of step S6 in FIG. 4, and associates the calculated matching result evaluation value A21 with the matching result M21.

The matching result evaluation unit 26 extracts Fp1 for the patient Q1 and Q2, Fp2 for the patient Q3 and Q5, and Fp3 for the patient Q4 from among the plurality of user priority scores calculated by the process of step S2 in FIG. 4 based on the matching result M22 obtained by the process of step S10 in FIG. 4. Next, the matching result evaluation unit 26 calculates a value obtained by summing the user priority scores extracted as described above as a matching result evaluation value A22 associated with the matching result M22. That is, the matching result evaluation unit 26 calculates the matching result evaluation value A22 by performing the process described above as the process of step S11 in FIG. 4, and associates the calculated matching result evaluation value A22 with the matching result M11.

Details of the third evaluation value calculation process described above will be described below.

The matching result evaluation unit 26 calculates a value obtained by summing the matching result evaluation value A11 calculated by the first evaluation value calculation process described above and the matching result evaluation value A21 calculated by the second evaluation value calculation process described above as a matching result evaluation value A31 associated with the matching results M11 and M21. That is, the matching result evaluation unit 26 calculates the matching result evaluation value A31 by performing the process described above as the process of step S11 in FIG. 4, and associates the calculated matching result evaluation value A31 with both the matching results M11 and M21.

The matching result evaluation unit 26 calculates a value obtained by summing the matching result evaluation value A12 calculated by the first evaluation value calculation process described above and the matching result evaluation value A22 calculated by the second evaluation value calculation process described above as a matching result evaluation value A32 associated with the matching results M12 and M22. That is, the matching result evaluation unit 26 calculates the matching result evaluation value A32 by performing the process described above as the process of step S11 in FIG. 4, and associates the calculated matching result evaluation value A32 with both the matching results M12 and M22.

By referring to the matching results and the matching result evaluation values stored in the DB 15, the matching result selection unit 27 determines whether or not there is an unprocessed priority in which the matching result evaluation value has not been calculated among all priorities possible to be changed by the process of step S9 in FIG. 4 (step S12 in FIG. 4).

When there is the unprocessed priority in which the matching result evaluation value has not been calculated among all priorities possible to be changed by the process of step S9 in FIG. 4 (S12: YES), the matching result selection unit 27 performs the process from step S9 to step S11 in FIG. 4 described above again. In addition, when the matching result evaluation value is calculated in all the priorities that can be changed by the process of step S9 in FIG. 4 (S12: NO), the matching result selection unit 27 performs the process of step S13 described below.

The matching result selection unit 27 selects one matching result evaluation value from among the matching result evaluation values stored in the DB 15, and acquires one matching result associated with the selected one matching result evaluation value as a final matching result (step S13 in FIG. 4). In other words, the matching result selection unit 27 includes a function as a matching result selection unit, and select, as a final matching result, either one of the matching result (first matching result) obtained by the matching process of step S5 (first matching process) in FIG. 4 and the matching result obtained by the matching process of step S10 (second matching process) in FIG. 4.

Here, for instance, in a case of performing the first evaluation value calculation process in step S6 in FIG. 4, the matching result selection unit 27 acquires 340 as the matching result evaluation value A11. Moreover, in a case of performing the first evaluation value calculation process is in step S11 in FIG. 4, the matching result selection unit 27 acquires 360 as the matching result evaluation value A12. Accordingly, based on a comparison result of the matching result evaluation value A11 and the matching result evaluation value A12, the matching result selection unit 27 acquires the matching result M12 associated with the matching result evaluation value A12 as the final matching result corresponding to the first evaluation value calculation process.

Moreover, for instance, in a case of performing the second evaluation value calculation process in step S6 in FIG. 4, the matching result selection unit 27 acquires 430 as the matching result evaluation value A21. In a case of performing the second evaluation value calculation process in step S11 in FIG. 4, the matching result selection unit 27 acquires 428 as the matching result evaluation value A22. Therefore, based on the comparison result of the matching result evaluation value A21 and the matching result evaluation value A22, the matching result selection unit 27 acquires the matching result M21 associated with the matching result A21 as the final matching result according to the second evaluation value calculation process.

Moreover, for instance, in a case of performing the third evaluation value calculation process in step S11 in FIG. 4, the matching result selection unit 27 acquires 770 as the matching result evaluation value A31 and also acquires 788 as the matching result evaluation value A32. Accordingly, based on the comparison result of the matching result evaluation value A31 and the matching result evaluation value A32, the matching result selection unit 27 acquires a matching result M32 associated with the matching result evaluation value A32 as the final matching result corresponding to the third evaluation value calculation process.

According to the present example embodiment, for instance, in a case where the first, second, and third evaluation value calculation processes are performed in step S11 in FIG. 4, the matching result selection unit 27 further performs a process for selecting one of the final matching results M12, M21, and M32 obtained as described above, as an optimal matching result.

The matching result output unit 28 outputs (sends) the final matching result obtained by the process of step S13 in FIG. 4 to the corresponding user terminal device 200 and the corresponding institution terminal device 300 (step S14 in FIG. 4).

The final matching result transmitted from the server device 100 is presented to the patient through a display screen or the like of the corresponding user terminal device 200, and is presented to the medical personnel through the display screen or the like of the corresponding institution terminal device 300.

Note that a series of processes in FIG. 4 is also applied in a similar manner in a case where the user threshold value Ft is set to 0 points, and the institution threshold value Ut is set to a value greater than 0 points. Moreover, the series of processes in FIG. 4 also applied in a similar manner in a case where the user threshold value Ft is set to a value greater than 0 points and the institution threshold value Ut is set to a value greater than 0 points.

Accordingly, in a case where the institution threshold value Ut is set to a value greater than 0 and the user threshold value Ft is set to 0, by the process of step S9 in FIG. 4, it is possible for the priority setting unit 24 to set priorities (fourth priorities) in which the institution priorities (second priorities) set in the process of step S4 in FIG. 4 are interchanged.

In addition, in a case where the institution threshold value Ut is set to a value greater than 0 and the user threshold value Ft is set to 0, in step S10 in FIG. 4, it is possible for the matching process unit 25 to perform the matching process based on combinations of the institution priorities (fourth priorities) changed by the process of step S9 in FIG. 4 and the user priorities (first priorities) set by the process of step S4 in FIG. 4.

Moreover, in a case where the user threshold value Ft is also set to a value greater than 0 and the institution threshold value Ut is set to a value greater than 0, by the process of step S9 in FIG. 4, it is possible for the priority setting unit 24 to set the priorities (third priorities) in which the user priorities (first priorities) set in the process of step S4 in FIG. 4 are interchanged, and to also set the priorities (fourth priorities) in which the institution priorities (second priorities) set in the process of step S4 in FIG. 4 are interchanged.

Furthermore, in a case where the user threshold value Ft is set to a value greater than 0 and the institution threshold value Ut is set to a value greater than 0, in step S10 in FIG. 4, it is possible for the matching process unit 25 to perform the matching process based on combinations of the user priorities (third priorities) changed by the process of step S9 in FIG. 4 and the institution priorities (the fourth priorities).

As described above, according to the present example embodiment, for instance, even in a case where a user who desires to be served by one of a large number of institutions considers two or more different institutions to be of roughly similar level of preference, it is possible to present an appropriate matching result to the user. Moreover, according to the present example embodiment, for instance, in a case where the institution which provides a service to a large number of users is able to accept two or more different users with roughly similar level of preference, it is possible to present an appropriate matching result to an employee or the like of the institution. As a result, according to the present example embodiment, it is possible to present an appropriate matching result in consideration of the preferences of both the user receiving the service and the provider of the service.

### Second Example Embodiment

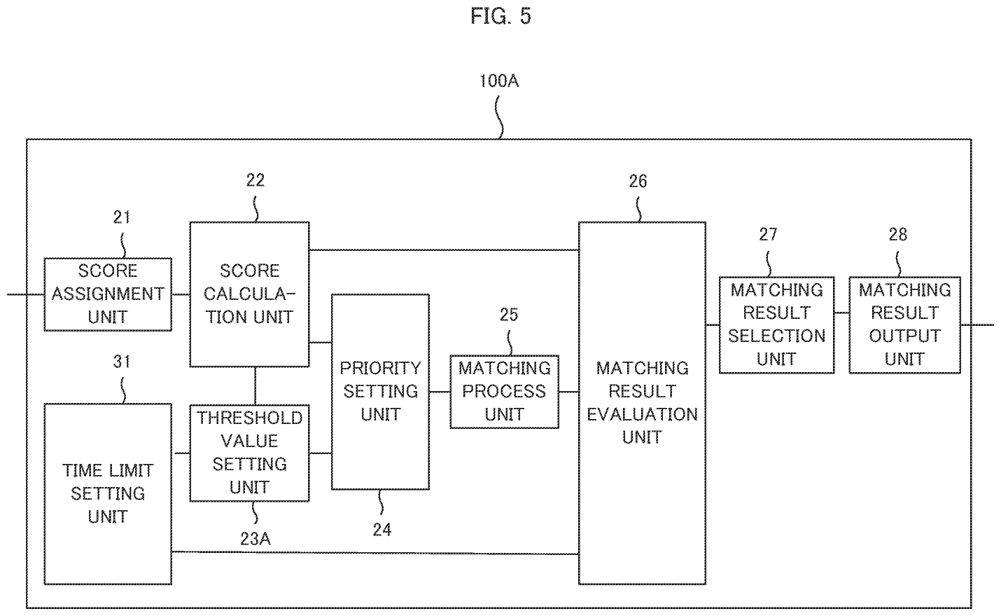

FIG. 5 is a block diagram illustrating a functional configuration of a server device according to a second example embodiment. In the following, for simplicity, specific explanations regarding parts to which the previously described processes and the like can be applied will be omitted as appropriate.

In the present example embodiment, the matching system 1 includes a server device 100A, the plurality of user terminal devices 200, and the plurality of institution terminal devices 300. The server device 100A includes a hardware configuration similar to the server device 100. As depicted in FIG. 5, the server device 100A includes the score assignment unit 21, the score calculation unit 22, a threshold value setting unit 23A, the priority setting unit 24, the matching process unit 25, the matching result evaluation unit 26, the matching result selection unit 27, the matching result output unit 28, and a time limit setting unit 31.

The threshold value setting unit 23A can set the user threshold value Ft and the institution threshold value Ut in a similar manner to the threshold value setting unit 23. Moreover, the threshold value setting unit 23A gradually increases the threshold value of one of the user threshold value Ft and the institution threshold value Ut from 0 points within a time limit TL to be described later. Incidentally, according to the present example embodiment, the threshold value setting unit 23A may gradually increase the threshold value from predetermined points other than 0 points within the time limit TL.

The time limit setting unit 31 sets the time limit TL concerning an execution of the matching process. The time limit setting unit 31 measures an elapsed time since the matching process has started, stops at least one of a process concerning the change of the threshold value in the threshold value setting unit 23A and a process concerning the calculation of the matching result evaluation value by the matching result evaluation unit 26 in a case where the measured elapsed time exceeds the time limit TL, and then causes the matching result selection unit 27 to perform a process concerning an acquisition of the final matching result.

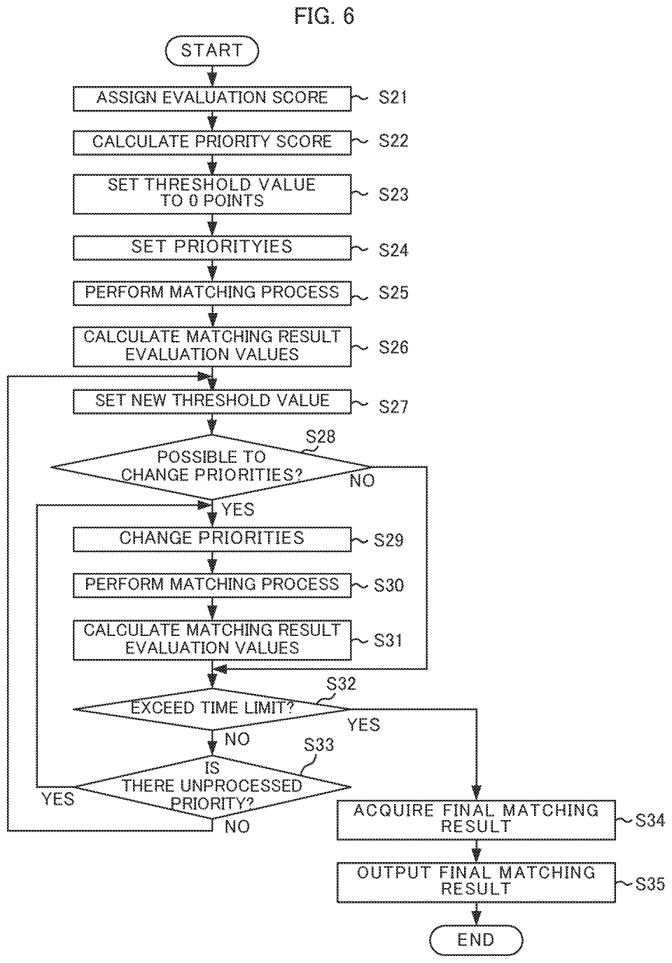

Next, operations of the present example embodiment will be described with reference to a flowchart in FIG. 6. In the following description of the present example embodiment, unless otherwise noted, the time limit setting unit 31 measures the elapsed time according to the execution of the matching process upon a timing of a start of a process represented in the flowchart in FIG. 6. Moreover, in the following description of the present example embodiment, a case will be described in which the time limit setting unit 31 stops the process concerning the calculation of the matching result evaluation values by the matching result evaluation unit 26 and then causes the process concerning the acquisition of the final matching result by the matching result selection unit 27. FIG. 6 is a flowchart for explaining an operation and the like of the server device according to the second example embodiment.

As a process of step S21 in FIG. 6, the score assignment unit 21 performs the same process as that of step S1 in FIG. 4, and assigns the institution evaluation scores Fe corresponding to respective user desired items and the user evaluation scores Ue corresponding to respective institution desired items.

The score calculation unit 22 calculates the user priority scores Fp and the institution priority scores Up by performing the same process as that of step S2 in FIG. 4 as a process of step S22 in FIG. 6.

The threshold value setting unit 23A sets the user threshold value Ft and the institution threshold value Ut to 0 points by performing the same process as that of step S3 in FIG. 4 as a process of step S23 in FIG. 6.

As a process of step S24 in FIG. 6, by performing the same process as that of step S4 in FIG. 4, the priority setting unit 24 sets the priorities in which the user priority scores Fp are ordered in the descending order as the user priorities, and sets the priorities ordered in which the institution priority scores Up are ordered in the descending order as the institution priorities.

The matching process unit 25 acquires the matching result corresponding to the user priorities and the institution priorities set by the process of step S24 in FIG. 6 by performing the same process as step S5 in FIG. 4 as a process of step S25 in FIG. 6. Moreover, the matching process unit 25 stores the matching result acquired by the process of step S25 in FIG. 6 in the DB 15.

As a process of step S26 in FIG. 6, the matching result evaluation unit 26 calculates each matching result evaluation value corresponding to the matching result obtained by the process of step S25 in FIG. 6, by performing the same process as that of step S6 in FIG. 4. Moreover, the matching result evaluation unit 26 associates the matching result evaluation values calculated by the process of step S26 in FIG. 6 with the matching result stored in the DB 15 by the process of step S25 in FIG. 6.

The threshold value setting unit 23A sets a new threshold value by increasing one of the user threshold value Ft and the institution threshold value Ut from 0 points (step S27 in FIG. 6).

Here, in the following explanation of the present example embodiment, unless otherwise specified, a case will be described in which by a process of step S27 in FIG. 6, the user threshold value Ft is incrementally increased by one point from 0 points (an initial setting value) while the institution threshold value Ut is maintained at 0 points.

The priority setting unit 24 performs a determination concerning whether or not it is possible to change the user priorities set in step S24 in FIG. 6 to the other priorities based on the plurality of user priority scores Fp calculated by the process of step S22 in FIG. 6 and the user threshold value Ft set by the process of step S27 in FIG. 6 (step S28 in FIG. 6).

Specifically, in step S28 in FIG. 6, the priority setting unit 24 detects whether or not there are the user priority scores of which each difference of scores falls in the range of one point or less among the plurality of user priority scores Fp calculated by the process of step S22 in FIG. 6.

When it is not possible to change the user priorities set in step S24 in FIG. 6 to other priorities (S28: NO), the priority setting unit 24 causes the matching result selection unit 27 to perform a process of step S33 in FIG. 6 described below within the time limit TL set by the time limit setting unit 31. Moreover, when the user priorities set in step S24 in FIG. 6 can be changed to another priority (S28: YES), the priority setting unit 24 performs a process of step S29 in FIG. 6 which will be described later.

Specifically, in a case where there are no user priority scores Fp for which each difference of scores falls in the range of 1 point or less among the plurality of user priority scores Fp calculated by the process of step S22 in FIG. 6, the priority setting unit 24 determines that it is not possible to change the user priorities set in step S24 in FIG. 6 to other priorities, and causes the matching result selection unit 27 to perform the process of step S33 in FIG. 6 described below in the limit time TL set by the limit time setting unit 31. Moreover, in a case where there are the user priority scores of which each difference of scores falls in the range of 1 point or less among the plurality of user priority scores Fp calculated by the process of step S22 in FIG. 6, the priority setting unit 24 determines that it is possible to change the user priorities set in step S24 in FIG. 6 to other priorities, and performs the process of step S29 in FIG. 6 described below.

By mutually interchanging the priorities of the institutions corresponding to the respective user priority scores Fp of which each difference of scores falls in the range of the user threshold value Ft set by the process of step S27 in FIG. 6 among the plurality of user priority scores Fp calculated by the process of step S22 in FIG. 6, the priority setting unit 24 changes the user priority currently being set to an unprocessed priority in which the calculation of the matching result evaluation values by a process of step S31 in FIG. 6 described below has not been performed (step S29 in FIG. 6).

That is, in a case where the user threshold value Ft is gradually increased from the initial setting value and the institution threshold value Ut is set to 0, the priority setting unit 24 iteratively performs the setting of the user priorities by the process of step S29 in FIG. 6 in a period until the elapsed time concerning the matching process exceeds the time limit TL.

The matching process unit 25 performs the matching process similar to that in the first example embodiment based on the user priorities changed by the process of step S29 in FIG. 6 and the institution priorities set by the process of step S24 in FIG. 6 (step S30 in FIG. 6). Moreover, the matching process unit 25 stores the matching result obtained by a process of step S30 in FIG. 6 in the DB 15.

That is, in a case where the user threshold value Ft is gradually increased from the initial setting value and the institution threshold value Ut is set to 0, the matching process unit 25 performs the process (first matching process) of step S25 in FIG. 6 in the period until the elapsed time concerning the matching process exceeds the time limit TL, and repeats the process (second matching process) of step S30 in FIG. 6 based on combinations of the user priorities changed by the process of step S29 in FIG. 6 and the institution priorities set by the process of step S24 in FIG. 6.

Note that the matching process unit 25 in the present example embodiment performs the process of step S30 in FIG. 6 for combinations of the user priorities and the institution priorities for which the matching result has not been obtained. That is, the matching process unit 25 in the present example embodiment does not perform the process of step S30 in FIG. 6 again for the combinations of the user priorities and the institution priorities for which the matching result has been obtained.

The matching result evaluation unit 26 calculates each matching result evaluation value by performing a similar process to that in the first example embodiment based on the plurality of user priority scores Fp and the plurality of institution priority scores Up calculated by the process of step S22 in FIG. 6 and the matching result obtained by the process of step S30 in FIG. 6 (step S31 in FIG. 6). Moreover, the matching result evaluation unit 26 associates the matching result evaluation values calculated by the process of step S31 in FIG. 6 with the matching result stored in the DB 15 by the process of step S30 in FIG. 6.

Note that the matching result evaluation unit 26 in the present example embodiment performs the process of step S31 in FIG. 6 for combinations of the user priorities and the institution priorities for which the matching result evaluation values have not been calculated. That is, the matching result evaluation unit 26 in the present example embodiment does not perform the process of step S31 in FIG. 6 again for the combinations of the user priorities and institution priorities of the matching result evaluation values which have been calculated.

The time limit setting unit 31 determines whether or not the elapsed time concerning the execution of the matching process in the server device 100A exceeds the time limit TL (step S32 in FIG. 6).

When the elapsed time concerning the execution of the matching process in the server device 100A is within the time limit TL (S32: NO), the time limit setting unit 31 causes the matching result selection unit 27 to perform the process of step S33 in FIG. 6 described below. In addition, when the elapsed time concerning the execution of the matching process in the server device 100A exceeds the time limit TL (S33: YES), the time limit setting unit 31 causes the matching result selection unit 27 to perform a process of step S34 in FIG. 6 described below.

By referring to the matching result and the matching result evaluation values stored in the DB 15, the matching result selection unit 27 determines whether or not there is an unprocessed priority in which the matching result evaluation value has not been calculated among all priorities possible to be changed by the process of step S29 in FIG. 6 (step S33 in FIG. 6).

When there is the unprocessed priority in which the matching result evaluation value has not been calculated among all priorities possible to be changed by the process of step S29 in FIG. 6 (S33: YES), the matching result selection unit 27 performs the process from step S29 to step S31 in FIG. 6 described above, again. Moreover, when the matching result evaluation values in all priorities possible to be changed by the process of step S29 in FIG. 6 are calculated (S33: NO), the matching result selection unit 27 performs the processes after step S27 in FIG. 6 again.

Here, in subsequent descriptions in the present example embodiment, the specific examples described above for the user priority scores and the institution priority scores in the first example embodiment will be referred to.

First, in a case where the user threshold value Ft is set to 0 by the process of step S23 in FIG. 6, the matching result M11 and the matching result evaluation value A11 or the matching result M21 and the matching result evaluation value A21 are stored in the DB 15.

Next, in a case where the user threshold value Ft is increased from 0 to 1 by the process of step S27 in FIG. 6, since there is no combination of scores corresponding to the determination condition of step S28 in FIG. 6 among the user priority scores for the patients Q1 to Q5, it is determined that the user priorities set in step S24 in FIG. 6 is not possible to be changed to other priorities. Accordingly, in a case where the user threshold value Ft is increased from 0 to 1 by the process of step S27 in FIG. 6 and it is in the time limit TL set by the time limit setting unit 31, the process is performed in an order of step S27→step S28→step S32→step S33→step S27 . . . , so that a new matching result and new matching result evaluation values are not stored in the DB 15.

Subsequently, in a case where the user threshold value Ft is increased from 1 point to 2 points by the process of step S27 in FIG. 6, since a combination of the user priority score Fp1 (=88 points) and Fp2 (=90 points) for the patient Q1 corresponds to the determination condition of step S28 in FIG. 6, it is determined that the user priorities set in step S24 in FIG. 6 is possible to be changed to other priorities. Therefore, in a case where the user threshold value Ft is increased from 1 point to 2 points by the process of step S27 in FIG. 6 and it is in the time limit TL set by the time limit setting unit 31, after the processes from step S27 to step S33 are sequentially performed, the process of step S27 is performed again. In addition, in a case where the user threshold value Ft is set to two points by the process of step S27 in FIG. 6, the matching result M12 and the matching result evaluation value A12, or the matching result M22 and the matching result evaluation value A22 are stored in the DB 15.

Subsequently, in a case where the user threshold value Ft is increased from 2 points to 3 points by the process of step S27 in FIG. 6, it is determined that the user priorities set in step S24 in FIG. 6 are possible to be changed to other priorities. Accordingly, in a case where the user threshold value Ft is increased from 2 points to 3 points by the process of step S27 in FIG. 6 and it is in the lime limit TL set by the time limit setting unit 31, the processes from step S27 to step S33 in FIG. 6 are sequentially performed. However, according to the specific examples of the user priority scores and the institution priority scores described above in the first example embodiment, the matching result and the matching result evaluation values when the user threshold value Ft is set to 3 points match with the matching result and the matching result evaluation values when the user threshold value Ft is set to 2 points. Therefore, in a case where the user threshold value Ft is increased from 2 to 3 by the process of step S27 in FIG. 6, a new matching result and the new matching result evaluation values are not stored to the DB 15 because the processes of step S30 and step S31 in FIG. 6 are not performed.

After that, the new matching result and the new matching result evaluation values are not stored in the DB 15 until the user threshold value Ft reaches 10 points.

Then, in a case where the user threshold value Ft reaches 10 points gradually increased, in the scores described above as the specific examples of the first example embodiment, because a large number of combinations of scores corresponding to the determination condition of step S28 in FIG. 6 exist, the number of times the process from step S29 to step S31 is repeated is enormous. Therefore, in the present example embodiment, a transition from step S32 to step S34 in FIG. 6 may be performed before all matching results and matching result evaluation values corresponding to the user threshold value Ft=10 points are stored in the DB 15.

That is, in the present example embodiment, even in a case where all matching results and matching result evaluation values corresponding to one user threshold value Ft are not stored in the DB 15, when the time limit TL set by the time limit setting unit 31 is exceeded, the iterative process from step S29 to step S31 in FIG. 6 is interrupted.

According to the present example embodiment, in a case where the transition from step S32 to step S34 in FIG. 6 is performed, the matching results and the matching result evaluation values according to the process performed within the time limit TL set by the time limit setting unit 31 are stored in the DB 15. Therefore, in the following, in response to the user threshold value Ft that is set to 10 points, the transaction from step S32 to step S34 in FIG. 6 is performed, one of the matching results corresponding to the user threshold value Ft=10 points is stored in the DB 15 as a matching result MRX, and matching result evaluation value ARX is associated with the matching result MRX.

Furthermore, in the present example embodiment, as the determination condition of step S32 in FIG. 6, instead of the time limited TL, for instance, the number of matching result evaluation values stored in the DB 15 in accordance with the process from step S29 to step S31 in FIG. 6, and the number of settings of the threshold values set by the process of step S27 in FIG. 6 may be used.

Moreover, in the present example embodiment, even in a case where the time limited TL is exceeded, the processes from step S29 to step S31 in FIG. 6 may be repeated until all matching results and matching result evaluation values corresponding to one user threshold value Ft are stored in the DB 15.

Among the matching result evaluation values stored in the DB 15 (calculated within the time limit TL), the matching result selection unit 27 excludes one or more matching evaluation values (including the matching result evaluation values ARX) corresponding to the most recent user threshold value Ft in which the process is interrupted by the time limit setting unit 31 from process targets concerning the selection of the final matching result. Incidentally, in a case where by repeating the process from step S29 to step S31 in FIG. 6, when all matching results and matching result evaluation values corresponding to the most recent user threshold value Ft are stored in the DB 15, the matching result evaluation value is not excluded from targets to be processed concerning the selection of the final matching result. Next, the matching result selection unit 27 selects one matching result evaluation value from among the matching result evaluation values which are stored in the DB 15 and not excluded from the process targets, and acquires one matching result associated with the one matching result evaluation value selected as the final matching result (step S34 in FIG. 6).

The matching result output unit 28 outputs (sends) the final matching result obtained by the process of step S13 in FIG. 4 to the user terminal device 200 and institution terminal device 300 (step S35 in FIG. 6).

The final matching result transmitted from the server device 100A is presented to the patient through a display screen or the like of the user terminal device 200 and is presented to the medical personnel through a display screen or the like of the institution terminal device 300.

Note that a series of processes in FIG. 6 is applied substantially the same as that even in a case where the user threshold value Ft is maintained at 0 points, while the institution threshold value Ut is gradually increased from 0 points (default setting value).

Therefore, in a case where the institution threshold value Ut is gradually increased from the initial setting value and the user threshold value Ft is set to 0, in a period until an elapsed time concerning the execution of the matching process exceeds the time limit TL, the priority setting unit 24, it is possible to repeatedly set the institution priorities by the process of step S29 in FIG. 6.

In addition, in a case where the institution threshold value Ut is gradually increased from the default setting and the user threshold value Ft is set to 0, it is possible for the matching process unit 25 to perform the process (first matching process) of step S25 in FIG. 6 in the period until the elapsed time concerning the execution of the matching process exceeds the time limit TL, and to also iteratively perform the process (second matching process) of step S30 in FIG. 6 based on the combinations of the user priorities set by the process of step S24 in FIG. 6 and the institution priorities changed by the process of step S29 in FIG. 6.

As described above, according to the present example embodiment, for instance, by performing the matching process while sequentially increasing the user threshold value Ft, it is possible to easily present the matching result in accordance with the preference of the user. Moreover, according to the present example embodiment, for instance, by performing the matching process while sequentially increasing the institution threshold value Ut, it is possible to easily present the matching result in accordance with the preference of the institution. As a result, according to the present example embodiment, it is possible to present an appropriate matching result in consideration of the desires of both the users of the services and the providers of the service.

### Third Example Embodiment

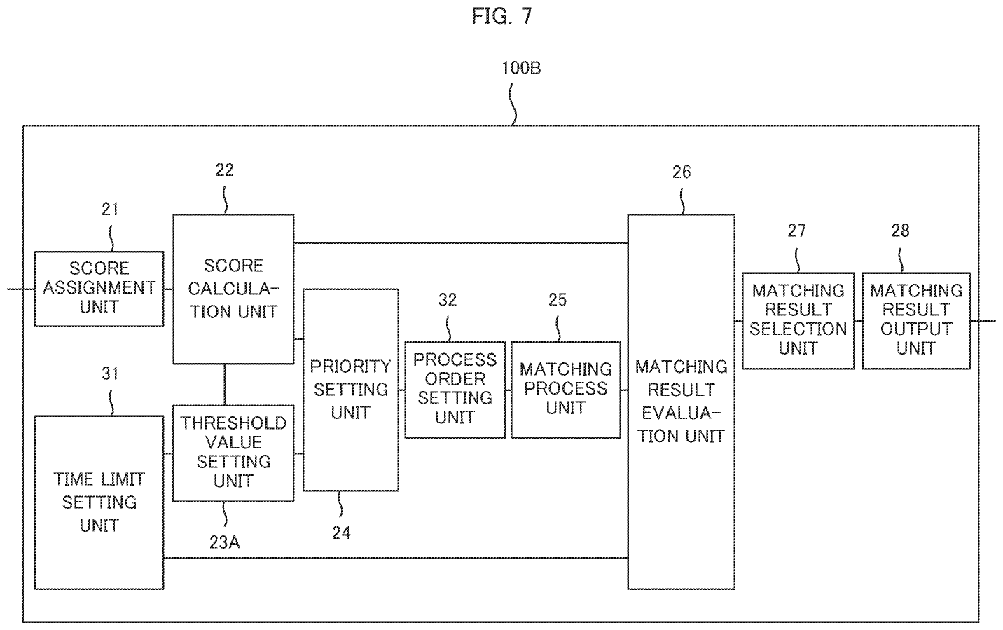

FIG. 7 is a block diagram illustrating a functional configuration of a server device according to a third example embodiment.

The matching system 1 according to the present example embodiment includes a server device 100B, the plurality of user terminal devices 200, and the plurality of institution terminal devices 300. The server device 100B includes the same hardware configuration as that of the server device 100. As depicted in FIG. 7, the server device 100B includes the score assignment unit 21, the score calculation unit 22, the threshold value setting unit 23A, the priority setting unit 24, the matching process unit 25, the matching result evaluation unit 26 the matching result selection unit 27, the matching result output unit 28, the time limit setting unit 31, and a process order setting unit 32.

The process order setting unit 32 includes a function as a process order setting unit, and sets a process order for performing the processes by the matching process unit 25 and the matching result evaluation unit 26 for each of the priorities acquired by the process of the priority setting unit 24.

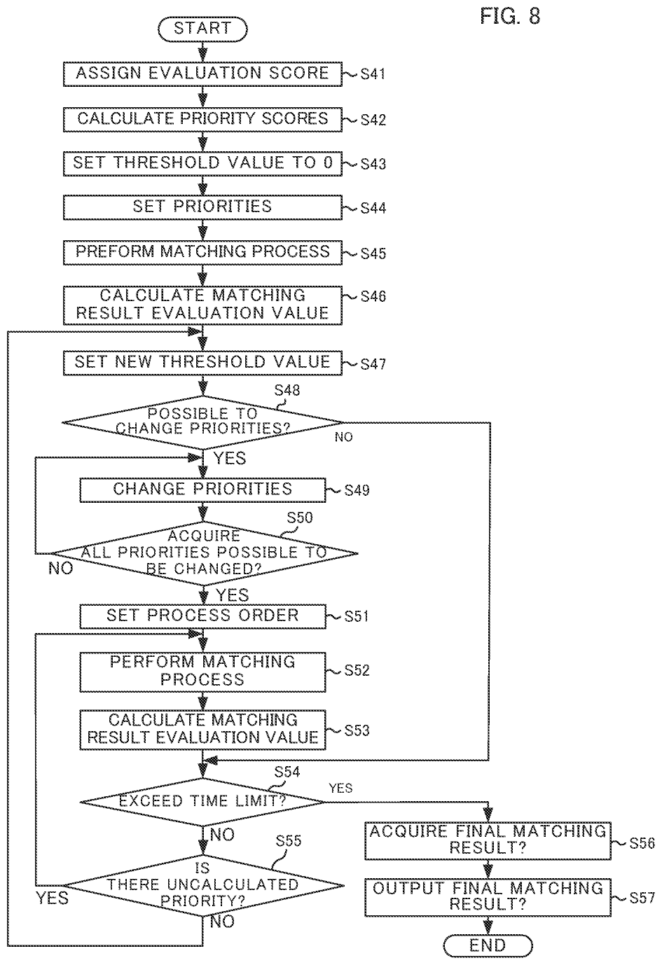

Subsequently, operations of the present example embodiment will be described with reference to the flowchart in FIG. 8. In the following descriptions of the present example embodiment, unless otherwise noted, it is assumed that the time limit setting unit 31 starts to measure the elapsed time concerning the execution of the matching process in the server device 100B from a timing at which the process depicted in the flowchart in FIG. 8 is started. Also, in the following descriptions of the present example embodiment, a case will be described in which the time limit setting unit 31 stops the process concerning the calculation of the matching result evaluation values in the matching result evaluation unit 26, and then causes the matching result selection unit 27 to perform the process concerning the acquisition of the final matching result. Moreover, in the following descriptions of the present example embodiment, among the user priority scores described above as the specific examples of the first example embodiment, an example will be described in which the user priority score Fp3 for the patient Q1 indicates 85 points and the user priority score Fp2 for the patient Q2 indicates 85 points. FIG. 8 is a flowchart for explaining an operation of the server device according to the third example embodiment.

As the process of step S41 in FIG. 8, the score assignment unit 21 performs the same process as that in step S1 in FIG. 4 to assign respective institution evaluation scores Fe corresponding to the plurality of user desired items and respective evaluation scores Ue corresponding to the plurality of institution desired items.

The score calculation unit 22 calculates the user priority scores Fp and the institution priority scores Up, by performing the same process as step S2 in FIG. 4 as the process of step S42 in FIG. 8.

The threshold value setting unit 23A sets the user threshold value Ft and the institution threshold value Ut to 0 points by performing the same process as that of step S3 in FIG. 4 as the process of step S43 in FIG. 8.

As the process of step S44 in FIG. 8, the priority setting unit 24 sets, by performing the same process as that of step S4 in FIG. 4, priorities in which the user priority scores Fp are arranged in the high order as the user priorities, and also sets priorities in which the institution priority scores Up are arranged in the high order as the institution priorities.

The matching process unit 25 acquires the matching result according to the user priorities and the institution priorities set by the process of step S44 in FIG. 8 by performing the same process as that of step S5 in FIG. 4, as the process of step S45 in FIG. 8. Also, the matching process unit 25 stores the matching result acquired by the process of step S45 in FIG. 8 in the DB 15.

As the process of step S46 in FIG. 8, the matching result evaluation unit 26 calculates the matching result evaluation values according to the matching result obtained by the process of step S45 in FIG. 8, by performing the same process as step S6 in FIG. 4. Moreover, the matching result evaluation unit 26 associates the matching result evaluation values calculated by the process of step S46 in FIG. 8 with the matching result stored in the DB 15 by the process of step S45 in FIG. 8.

The threshold value setting unit 23A sets a new threshold value by increasing one of the user threshold value Ft and the institution threshold value Ut from 0 points (step S47 in FIG. 8).

Here, in the following descriptions of the present example embodiment, unless otherwise noted, an example of a case will be described in which by the process of step S47 in FIG. 8, the user threshold value Ft is increased from 0 to 5 points while the institution threshold value Ut is maintained at 0 points.

The priority setting unit 24 performs a determination concerning whether or not it is possible to change the user priorities set in step S44 in FIG. 8 to the other priorities based on the plurality of user priority scores Fp calculated by the process of step S42 in FIG. 8 and the user threshold value Ft set by the process of step S47 in FIG. 8 (step S48 in FIG. 8).

Specifically, the priority setting unit 24 detects whether or not there are the user priority scores Fp of which each difference of scores falls in the range of 5 points or less among the plurality of user priority scores Fp calculated by the process of step S42 in FIG. 8.

When it is not possible to change the user priorities set in step S44 in FIG. 8 to change other priorities (S48:NO), the priority setting unit 24 causes the matching result selection unit 27 to perform the process of step S55 in FIG. 8 described below to the matching result selection unit 27 within the time limit TL. Moreover, when it is possible to change the user priorities set in step S44 in FIG. 8 to change other priorities (S48: YES), the priority setting unit 24 performs a process of step S49 in FIG. 8.

By mutually interchanging priorities of the institutions corresponding to respective user priority scores Fp of which each difference of scores falls in the range of the user threshold value Ft set by the process of step S47 in FIG. 8 among the plurality of user priority scores Fp calculated by the process of step S42 in FIG. 8, the priority setting unit 24 changes a user priority currently being set to an unset priority except those already set in a previous process (step S49 in FIG. 8). Moreover, the priority setting unit 24 stores the user priorities set by the process of step S49 in FIG. 8 in the DB 15.

By referring to the user priorities stored in the DB 15, the priority setting unit 24 determines whether or not all priorities possible to be set are acquired by the process of step S49 in FIG. 8 (step S50 in FIG. 8).

When all priorities are not acquired by the process of step S49 in FIG. 8 (S50: NO), the priority setting unit 24 performs the process of step S49 in FIG. 8, again. On the other hand, when all priorities are acquired by the process of step S49 (S50: YES), the priority setting unit 24 causes the process order setting unit 32 to perform a process of step S51 in FIG. 8 which will be described later.

Here, according to the present example embodiment, since priorities of institutions P1, P2 and P3 are mutually interchangeable due to the user priority scores Fp1, Fp2 and Fp3 for patient Q1 being in the range of 5 points or less, it is possible to set priorities of six patterns as the user priorities for the patient Q1. Moreover, according to the present example embodiment, since the user priority scores Fp1 and Fp2 for the patient Q2 being in the range of 5 points or less and priorities for the institutions P1 and P2 are mutually interchangeable, it is possible to set priorities of the two patterns for the patient Q2 as the user priorities.

Therefore, according to the present example embodiment, in a state where the user threshold value Ft is set to 5 points, the priority setting unit 24 iteratively performs the processes of step S49 and step S50 in FIG. 8, and priorities of 12 patterns are thus stored as the user priorities of the institutions P1 to P3 for the patients Q1 to Q5 in the DB 15.

The process order setting unit 32 sets a process order for performing the processes of step S52 and step S53 in FIG. 8 described later with respect to each of the user priorities stored in the DB 15 through the processes of step S49 and step S50 in FIG. 8 (step S51 in FIG. 8). Moreover, the process order setting unit 32 stores the process order set as described above in the DB 15.

Specifically, the process order setting unit 32 acquires the number of patients in each of the institutions which are first in the priorities based on the user priorities stored in the DB 15, and sets the process order to perform the processes of step S52 and the step S53 in FIG. 8 in an ascending order of the acquired number of patients.

Here, according to the priorities of 12 patterns, the priorities of the institutions P1 to P3 for the patient Q1 are ordered as a first highest priority, and the priorities of the institutions P1 to P2 for the patient Q2 are ordered as a first highest priority.

Therefore, according to the priorities of the 12 patterns described above, it is estimated that the patients Q1, Q2, Q3, and Q5 (4 patients) are aware that the institution P1 may be their first choice. Moreover, according to the priorities of the 12 patterns described above, it is estimated that the patients Q1, Q2 and Q4 (3 patients) are aware that the institution P2 may be their first choice. Furthermore, according to the priorities of the 12 patterns described above, it is estimated that the patient Q1 (1 patient) is aware that the institution P3 may be the first choice.

Therefore, for instance, in a case where the priorities of the 12 patterns described above are stored in the DB 15, the process order setting unit 32 sets the process order so that a first priority represented by the institution P3→the institution P2→the institution P1 is processed among the use priorities for the patient Q1. Next, the process order setting unit 32 sets the process order (from the first to the 12th) with respective to the priorities of the 12 patterns described above by performing the process of step S51 in FIG. 8, and stores the set process order in the DB 15.

Note that in the present example embodiment, the processes from step S49 to step S51 in FIG. 8 may be performed for the user priorities in consideration of the number of acceptable patients for each institution. Moreover, in the present example embodiment, for instance, in a case of gradually increasing the institution threshold value Ut while maintaining the user threshold value Ft to 0 points, the processes from step S49 to step S51 in FIG. 8 for the institution priorities may be performed. Furthermore, in the present example embodiment, for instance, in a case of alternately gradually increasing the user threshold value Ft and the institution threshold value Ut, the process from step S49 to step S51 in FIG. 8 for both the user priorities and the institution priorities may be performed.

The matching process unit 25 performs the matching process similar to the second example embodiment based on the process order set by the process of step S51 in FIG. 8 (step S52 in FIG. 8). Moreover, the matching process unit 25 stores the matching result obtained by a process of step S52 in FIG. 8 in the DB 15.

Note that the matching process unit 25 of the present example embodiment performs the process of step S52 in FIG. 8 for the combinations of the user priorities and the institution priorities for which the matching result has not been obtained. That is, the matching process unit 25 of the present example embodiment does not perform the process of step S52 in FIG. 8 again for the combinations of the user priorities and the institution priorities for which the matching result has been obtained.

The matching result evaluation unit 26 calculates the matching result evaluation values by performing a similar process to that in the second example embodiment based on the plurality of user priority scores Fp and the plurality of institution priority scores Up calculated by the process of step S42 in FIG. 8 and the matching result obtained by the process of step S52 in FIG. 8 (step S53 in FIG. 8). Moreover, the matching result evaluation unit 26 associates the matching result evaluation values calculated by a process of step S53 in FIG. 8 with the matching result stored in the DB 15 by the process of step S52 in FIG. 8.

The matching result evaluation unit 26 of the present example embodiment performs the process of step S53 in FIG. 8 for the combinations of the user priorities and the institution priorities for which the matching result evaluation values have not been calculated. That is, the matching result evaluation unit 26 of the present example embodiment does not perform the process of step S53 in FIG. 8 again for the combinations of the user priorities and institution priorities of the matching result evaluation values which are calculated.

The time limit setting unit 31 determines whether or not the elapsed time concerning the execution of the matching process in the server device 100B exceeds the time limit TL (step S54 in FIG. 8).

Upon the elapsed time concerning the execution of the matching process in the server device 100B that is within the time limit TL (S54:NO), the time limit setting unit 31 causes the matching result selection unit 27 to perform a process of step S55 in FIG. 8 described below. On the other hand, upon the elapsed time concerning the execution of the matching process in the server device 100B that exceeds the time limit TL (S54: YES), the time limit setting unit 31 causes the matching result selection unit 27 to perform a process of step S56 in FIG. 8 described below.

Referring to the DB 15, the matching result selection unit 27 determines whether or not there is an unprocessed priority in which the matching result evaluation values have not been calculated, based on the process order set by the process of step S51 in FIG. 8 (step S55 in FIG. 8).

When there is the unprocessed priority order in which the matching result evaluation values have not been calculated in the process order set by the process of step S51 in FIG. 8 (S55: YES), the matching result selection unit 27 performs the process of step S52 and step S53 in FIG. 8 described above again. On the other hand, in the priorities of all the patterns included in the process order set by the process of step S51 in FIG. 8, when the matching result evaluation values are calculated (S55: NO), the matching result selection unit 27 performs the process after step S47 in FIG. 8 again.

The matching result selection unit 27 selects one matching result evaluation value from among the matching result evaluation values stored in the DB 15, and acquires one matching result associated with the selected matching result evaluation value as a final matching result (step S56 in FIG. 8).

In the present example embodiment, due to the setting of the process order by the process of step S51 in FIG. 8, the matching result evaluation values ARX can be calculated as values with validity. For this reason, in the present example embodiment, for instance, the matching result selection unit 27 performs the process of step S56 in FIG. 8 without excluding the matching result evaluation values ARX, so that the matching values MRX associated with the matching result evaluation values ARX may be acquired as the final matching result.

The matching result output unit 28 outputs (sends) the final matching result obtained by the process of step S56 in FIG. 8 to the corresponding user terminal device 200 and the corresponding institution terminal device 300 (step S57 in FIG. 8).

As described above, according to the present example embodiment, for instance, by setting the process order concerning the matching process based on the user priorities, it is possible to easily present the matching result in accordance with the desire of the user. Moreover, according to the present example embodiment, for instance, by setting the process order in accordance with the matching process based on the institution priorities, it is possible to easily present the matching result in accordance with the desire of the institution. As a result, according to the present example embodiment, it is possible to present an appropriate matching result in consideration of the desires of both the user of the service and the provision of the service.

### Fourth Example Embodiment

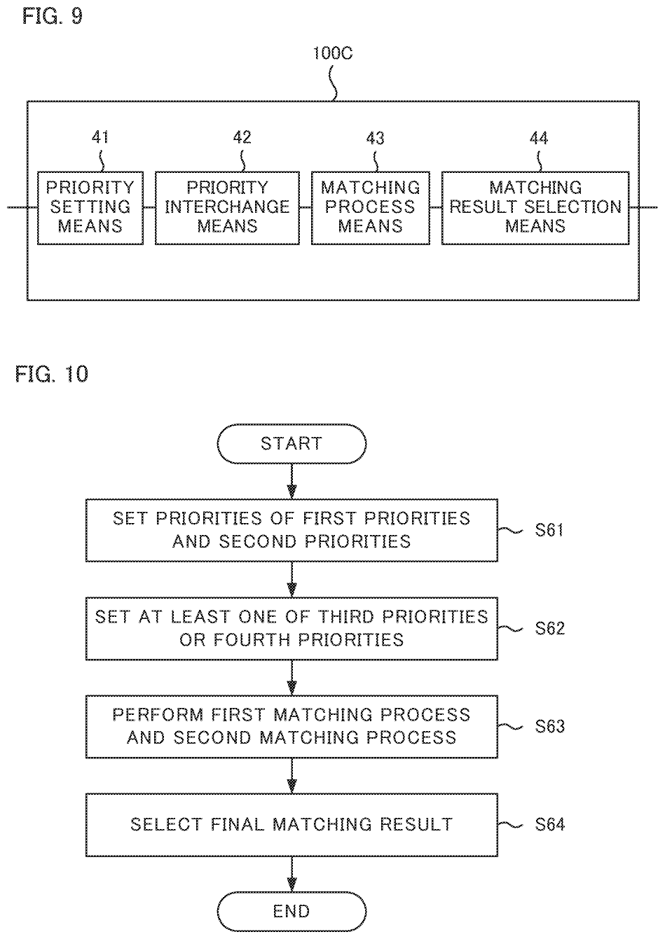

FIG. 9 is a block diagram illustrating a functional configuration of a server device according to a fourth example embodiment.

The matching system 1 according to the present example embodiment includes a server device 100C, the plurality of user terminal devices 200, and the plurality of institution terminal devices 300. Moreover, the server device 100C includes the same hardware configuration as that of the server device 100. Also, as depicted in FIG. 9, the server device 100C includes a priority setting means 41, a priority interchange means 42, a matching process means 43, and a matching result selection means 44.

FIG. 10 is a flowchart for explaining operations and the like of the server device according to the fourth example embodiment.

The priority setting means 41 sets the first priorities by arranging scores in the descending order which are scores assigned based on desired conditions of the plurality of users who receive a service and which are assigned to the plurality of providers for providing the service, and also sets the second priorities by arranging scores in the descending order which are scores assigned based on desired conditions of to the plurality of providers and which are assigned to the plurality of users (step S61 in FIG. 10).

The priority interchange means 42 sets at least one of the third priorities in which the priorities of the providers are interchanged based on the differences of the scores assigned in the first priorities and the fourth priorities in which the priorities of the users are interchanged based on the differences of the scores assigned in the second priorities (step S62 in FIG. 10).

As the matching process for matching the plurality of users and the plurality of providers, the matching process means 43 performs the first matching process based on the combinations of the first priorities and the second priorities and also performs the second matching process based on the combinations of the third priorities and the second priorities, the combinations of the first priorities and the fourth priorities, or the combinations of the third priorities and the fourth priorities (step S63 in FIG. 10).

The matching result selection means 44 selects either one of the first matching result obtained by the first matching process and the second matching result obtained by the second matching process as the final matching result (step S64 in FIG. 10).

According to the present example embodiment, it is possible to present the appropriate matching result in consideration of the desires of both the users of the service and the providers of the service.

A part or all of the example embodiments described above may also be described as the following supplementary notes, but not limited thereto.

### (Supplementary Note 1)

A matching device comprising:
a priority setting means configured to set first priorities by arranging scores in a descending order which are scores assigned based on desired conditions of a plurality of users who receive a service and which are assigned respectively to a plurality of providers serving the service, and also to set second priorities by arranging scores in the descending order which are scores assigned based on desired conditions of the plurality of providers and which are assigned respectively to the plurality of users;
a priority interchange means configured to set at least one of third priorities in which priorities of the plurality of providers are interchanged based on differences of the scores assigned in the first priorities and fourth priorities in which priorities of the plurality of users are interchanged based on differences of the scores assigned in the second priorities;
a matching process means configured to perform a first matching process based on combinations of the first priorities and the second priorities and also to perform a second matching process based on combinations of the third priorities and the second priorities, combinations of the first priorities and the fourth priorities, or combinations of the third priorities and the fourth priorities, as a matching process for matching the plurality of users and the plurality of providers; and
a matching result selection means configured to select, as a final matching result, one of a first matching result acquired by the first matching process and a second matching result acquired by the second matching process.

### (Supplementary Note 2)

The matching device according to supplementary note 1, wherein
the third priorities are set by interchanging priorities of respective providers for which the differences of the scores assigned in the first priorities are in a range of a first threshold value, and
the fourth priorities are set by interchanging priorities of respective users for which the differences of the scores assigned in the second priorities are in a range of a second threshold value.

### (Supplementary Note 3)

The matching device according to supplementary note 2, wherein
the priority interchange means sets the third priorities when the first threshold value is set to a greater value than 0 and the second threshold value is also set to 0, and
the matching process means performs the first matching process, and the second matching process which is based on the combinations of the third priorities and the second priorities when the first threshold value is set to a greater value than 0 and the second threshold value is set to 0.

### (Supplementary Note 4)

The matching device according to supplementary note 2, wherein
the priority interchange means repeats setting of the third priorities in a period until an elapsed time concerning an execution of the matching process exceeds a time limit, when the first threshold value gradually increases from an initial setting value and the second threshold value is also set to 0, and
the matching process means performs the first matching process in the period and also repeats the second matching process based on the combination of the third priorities and the second priorities, when the first threshold value gradually increases from the initial setting value and the second threshold value is also set to 0.

### (Supplementary Note 5)

The matching device according to supplementary note 4, further comprising a process order setting means configured to set a process order in a case of performing the second matching process based on the combinations of the third priorities and the second priorities.

### (Supplementary Note 6)

The matching device according to supplementary note 2, wherein
the priority interchange means sets the fourth priorities, when the second threshold value is set to the greater value than 0 and the first threshold value is also set to 0, and
the matching process means performs the first matching process, and the second matching process which is based on the combinations of the first priorities and the fourth priorities, when the second threshold value is set to the greater value than 0 and the first threshold value is set to 0.

### (Supplementary Note 7)

The matching device according to supplementary note 2, wherein
the priority interchange means repeats setting of the fourth priorities in the period until the elapsed time exceeds the time limit, when the second threshold value gradually increases from an initial setting value and the first threshold value is set to 0, and
the matching process means performs the first matching process and also repeats the second matching process based on the combinations of the first priorities and the fourth priorities in the period, when the second threshold value gradually increases from the initial setting value and the first threshold value is set to 0.

### (Supplementary Note 8)

The matching device according to supplementary note 7, further comprising a process order setting means configured to set a process order used in a case of performing the second matching process based on the combinations of the first priorities and the fourth priorities.

### (Supplementary Note 9)

A matching method comprising:
setting first priorities by arranging scores in a descending order which are scores assigned based on desired conditions of a plurality of users who receive a service and which are assigned respectively to a plurality of providers serving the service, and also setting second priorities by arranging scores in the descending order which are scores assigned based on desired conditions of the plurality of providers and which are assigned respectively to the plurality of users;
setting at least one of third priorities in which priorities of the plurality of providers are interchanged based on differences of the scores assigned in the first priorities and fourth priorities in which priorities of the plurality of users are interchanged based on differences of the scores assigned in the second priorities;
performing a first matching process based on combinations of the first priorities and the second priorities and also performing a second matching process based on combinations of the third priorities and the second priorities, combinations of the first priorities and the fourth priorities, or combinations of the third priorities and the fourth priorities, as a matching process for matching the plurality of users and the plurality of providers; and
selecting, as a final matching result, one of a first matching result acquired by the first matching process and a second matching result acquired by the second matching process.

### (Supplementary Note 10)

A recording medium storing a program, the program causing a computer to perform a process comprising:
setting first priorities by arranging scores in a descending order which are scores assigned based on desired conditions of a plurality of users who receive a service and which are assigned respectively to a plurality of providers serving the service, and also setting second priorities by arranging scores in the descending order which are scores assigned based on desired conditions of the plurality of providers and which are assigned respectively to the plurality of users;
setting at least one of third priorities in which priorities of the plurality of providers are interchanged based on differences of the scores assigned in the first priorities and fourth priorities in which priorities of the plurality of users are interchanged based on differences of the scores assigned in the second priorities;
performing a first matching process based on combinations of the first priorities and the second priorities and also performing a second matching process based on combinations of the third priorities and the second priorities, combinations of the first priorities and the fourth priorities, or combinations of the third priorities and the fourth priorities, as a matching process for matching the plurality of users and the plurality of providers; and
selecting, as a final matching result, one of a first matching result acquired by the first matching process and a second matching result acquired by the second matching process.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

12 Processor
22 Score calculation unit
23, 23A Threshold setting unit
25 Matching process unit
26 Matching result evaluation unit
27 Matching result selection unit
31 Time limit setting unit
32 Process order setting unit

What is claimed is:

1. A matching device comprising:
at least one memory configured to store instructions; and
at least one processor configured to execute the instructions to:
set first priorities by arranging scores in a descending order which are scores assigned based on desired conditions of a plurality of users who receive a service and which are assigned respectively to a plurality of providers serving the service, and also to set second priorities by arranging scores in the descending order which are scores assigned based on desired conditions of the plurality of providers and which are assigned respectively to the plurality of users;
a priority interchange means configured to set at least one of third priorities in which priorities of the plurality of providers are interchanged based on differences of the scores assigned in the first priorities and fourth priorities in which priorities of the plurality of users are interchanged based on differences of the scores assigned in the second priorities;
perform a first matching process based on combinations of the first priorities and the second priorities and also to perform a second matching process based on combinations of the third priorities and the second priorities, combinations of the first priorities and the fourth priorities, or combinations of the third priorities and the fourth priorities, as a matching process for matching the plurality of users and the plurality of providers; and
a matching result selection means configured to select, as a final matching result, one of a first matching result acquired by the first matching process and a second matching result acquired by the second matching process.

2. The matching device according to claim 1, wherein
the third priorities are set by interchanging priorities of respective providers for which the differences of the scores assigned in the first priorities are in a range of a first threshold value, and
the fourth priorities are set by interchanging priorities of respective users for which the differences of the scores assigned in the second priorities are in a range of a second threshold value.

3. The matching device according to claim 2, wherein
the priority interchange means-processor sets the third priorities when the first threshold value is set to a greater value than 0 and the second threshold value is also set to 0, and
the matching process means-processor performs the first matching process, and the second matching process which is based on the combinations of the third priorities and the second priorities when the first threshold value is set to a greater value than 0 and the second threshold value is set to 0.

4. The matching device according to claim 2, wherein
the priority interchange means-processor repeats setting of the third priorities in a period until an elapsed time concerning an execution of the matching process exceeds a time limit, when the first threshold value gradually increases from an initial setting value and the second threshold value is also set to 0, and
the matching process means-processor performs the first matching process in the period and also repeats the second matching process based on the combination of the third priorities and the second priorities, when the first threshold value gradually increases from the initial setting value and the second threshold value is also set to 0.

5. The matching device according to claim 4, wherein the processor is further comprising a process order setting means-configured to set a process order in a case of performing the second matching process based on the combinations of the third priorities and the second priorities.

6. The matching device according to claim 2, wherein
the priority interchange means-processor sets the fourth priorities, when the second threshold value is set to the greater value than 0 and the first threshold value is also set to 0, and
the matching process means-processor performs the first matching process, and the second matching process which is based on the combinations of the first priorities and the fourth priorities, when the second threshold value is set to the greater value than 0 and the first threshold value is set to 0.

7. The matching device according to claim 2, wherein
the priority interchange means processor repeats setting of the fourth priorities in the period until the elapsed time exceeds the time limit, when the second threshold value gradually increases from an initial setting value and the first threshold value is set to 0, and
the matching process means-processor performs the first matching process and also repeats the second matching process based on the combinations of the first priorities and the fourth priorities in the period, when the second threshold value gradually increases from the initial setting value and the first threshold value is set to 0.

8. The matching device according to claim 7, wherein the processor is further comprising a process order setting means-configured to set a process order used in a case of performing the second matching process based on the combinations of the first priorities and the fourth priorities.

9. A matching method comprising:
setting first priorities by arranging scores in a descending order which are scores assigned based on desired conditions of a plurality of users who receive a service and which are assigned respectively to a plurality of providers serving the service, and also setting second priorities by arranging scores in the descending order which are scores assigned based on desired conditions of the plurality of providers and which are assigned respectively to the plurality of users;
setting at least one of third priorities in which priorities of the plurality of providers are interchanged based on differences of the scores assigned in the first priorities and fourth priorities in which priorities of the plurality of users are interchanged based on differences of the scores assigned in the second priorities;
performing a first matching process based on combinations of the first priorities and the second priorities and also performing a second matching process based on combinations of the third priorities and the second priorities, combinations of the first priorities and the fourth priorities, or combinations of the third priorities and the fourth priorities, as a matching process for matching the plurality of users and the plurality of providers; and
selecting, as a final matching result, one of a first matching result acquired by the first matching process and a second matching result acquired by the second matching process.

10. A non-transitory computer-readable recording medium storing a program, the program causing a computer to perform a process comprising:
setting first priorities by arranging scores in a descending order which are scores assigned based on desired conditions of a plurality of users who receive a service and which are assigned respectively to a plurality of providers serving the service, and also setting second priorities by arranging scores in the descending order which are scores assigned based on desired conditions of the plurality of providers and which are assigned respectively to the plurality of users;
setting at least one of third priorities in which priorities of the plurality of providers are interchanged based on differences of the scores assigned in the first priorities and fourth priorities in which priorities of the plurality of users are interchanged based on differences of the scores assigned in the second priorities;
performing a first matching process based on combinations of the first priorities and the second priorities and also performing a second matching process based on combinations of the third priorities and the second priorities, combinations of the first priorities and the fourth priorities, or combinations of the third priorities and the fourth priorities, as a matching process for matching the plurality of users and the plurality of providers; and
selecting, as a final matching result, one of a first matching result acquired by the first matching process and a second matching result acquired by the second matching process.
